(19) United States
(12) Patent Application Publication
(10) Pub. No.: US 2013/0203790 A1
(43) Pub. Date: Aug. 8, 2013

(54) SULFUR-CONTAINING COMPOUNDS AS ANTI-PROLIFERATIVE AGENTS

(71) Applicants: Dheeraj RAJAN, Toronto (CA); Mark Otto BAERLOCHER, Barrie (CA)

(72) Inventors: Dheeraj RAJAN, Toronto (CA); Mark Otto BAERLOCHER, Barrie (CA)

(73) Assignee: 221076 ONTARIO INC., Toronto, ON (CA)

(21) Appl. No.: 13/544,969

(22) Filed: Jul. 9, 2012

Related U.S. Application Data
(60) Provisional application No. 61/506,202, filed on Jul. 11, 2011.

(51) Int. Cl. A61K 31/145; A61K 31/4409; A61K 31/4402; C07C 321/28; A61P 19/08; A61K 31/505; A61P 9/10; A61P 7/02; A61P 29/00; C07D 213/71; C07D 239/38

(52) U.S. Cl. 514/274; 546/294; 514/347; 564/440; 514/646; 544/318

(57) ABSTRACT

Novel sulphur-containing compounds and pharmaceutically acceptable salts thereof are disclosed, which have utility as anti-proliferative agents against mammalian cells. The invention provides a method for synthesizing the sulphur-containing compounds.

### FIELD OF THE INVENTION

This invention is directed towards novel sulphur-containing compounds and pharmaceutically acceptable salts thereof, which have utility as anti-proliferative agents against mammalian cells. The invention provides a method for synthesizing the sulphur-containing compounds.

### BACKGROUND OF THE INVENTION

The present invention provides novel compounds, novel compositions, methods of their use and methods of their manufacture, such compounds being generally pharmacologically useful as anti-platelet aggregation agents in various vascular pathologies. The aforementioned pharmacologic activities are useful in the treatment of mammals. At the present time, there is a need in the area of vascular therapeutics for such a blocking agent. By interfering with hemostasis, such therapy would decrease the morbidity and mortality of thrombotic disease.

Hemostasis is the spontaneous process of stopping bleeding from damaged blood vessels. Precapillary vessels contract immediately when cut. Within seconds, thrombocytes, or blood platelets, are bound to the exposed matrix of the injured vessel by a process called platelet adhesion. Platelets also stick to each other in a phenomenon known as platelet aggregation to form a platelet plug. This platelet plug can stop bleeding quickly, but it must be reinforced by the protein fibrin for long-term effectiveness, until the blood vessel tear can be permanently repaired by growth of fibroblasts, which are specialized tissue repair cells.

An intravascular thrombus (clot) results from a pathological disturbance of hemostasis. The thrombus can grow to sufficient size to block off arterial blood vessels. Thrombi can also form in areas of stasis or slow blood flow in veins. Venous thrombi can easily detach portions of themselves called emboli that travel through the circulatory system and can result in blockade of other vessels, such as pulmonary arteries. Thus, arterial thrombi cause serious disease by local blockade, whereas venous thrombi do so primarily by distant blockade, or embolization. These diseases include venous thrombosis, thrombophlebitis, arterial embolism, coronary and cerebral arterial thrombosis and myocardial infarction, stroke, cerebral embolism, kidney embolisms and pulmonary embolisms.

There is a need in the area of cardiovascular and cerebrovascular therapeutics for an agent that can be used in the prevention and treatment of thrombi, with minimal side effects, including unwanted prolongation of bleeding in other parts of the circulation while preventing or treating target thrombi. The compounds of the present invention meet this need in the art by providing therapeutic agents for the prevention and treatment of thrombi.

The compounds of the present invention show efficacy as antithrombotic agents by virtue of their ability to block fibrinogen from acting at its platelet receptor site and thus prevent platelet aggregation.

### SUMMARY OF THE INVENTION

The present invention relates to certain disuphides, and pharmaceutically acceptable salts thereof, having activity as anti-proliferative agents, to methods for their preparation, and to methods and pharmaceutical formulations for using these compounds in mammals (especially humans).

Because of their activity as anti-proliferative agents, the compounds of the present invention are useful for the treatment of a variety of conditions, including arterial or venous thrombosis, inflammation, bone degradation, malignancy (primary or secondary), cell aggregation-related conditions, thromboembolic disorders selected from thrombus or embolus formation, harmful platelet aggregation, re-occlusion following thrombolysis, reperfusion injury, restenosis, atherosclerosis, stroke, heart attack, peripheral arterial ischemia, myocardial infarction, and unstable angina.

### BRIEF DESCRIPTION OF THE FIGURES

FIG. 1 is a 1H-NMR spectrum of 4-methyldisulfanyl-pyridine, in accordance with one embodiment of the present invention.

FIG. 2 is a 1H-NMR spectrum of 2-methyldisulfanyl-pyridine, in accordance with one embodiment of the invention.

FIG. 3 is a 1H-NMR spectrum of 4-methyldisulfanyl-phenylamine, in accordance with one embodiment of the invention.

FIG. 4 is a 1H-NMR spectrum of 2-methyldisulfanyl-pyrimidine, in accordance with one embodiment of the invention.

FIG. 5 is a platelet inhibition assay using platelet rich plasma of 4-methyldisulfanyl-pyridine, in accordance with one embodiment of the present invention.

FIG. 6 is a platelet inhibition assay using platelet rich plasma of 2-methyldisulfanyl-pyridine, in accordance with one embodiment of the present invention.

FIG. 7 is a platelet inhibition assay using platelet rich plasma of 4-methyldisulfanyl-phenylamine, in accordance with one embodiment of the present invention.

FIG. 8 is a platelet inhibition assay using platelet rich plasma of 2-methyldisulfanyl-pyrimidine, in accordance with one embodiment of the present invention.

FIG. 9 is a platelet inhibition assay using whole blood of 4-methyldisulfanyl-pyridine, in accordance with one embodiment of the present invention.

FIG. 10 is a platelet inhibition assay using whole blood of 2-methyldisulfanyl-pyridine, in accordance with one embodiment of the present invention.

FIG. 11 is a platelet inhibition assay using whole blood of 4-methyldisulfanyl-phenylamine, in accordance with one embodiment of the present invention.

FIG. 12 is a platelet inhibition assay using whole blood of 2-methyldisulfanyl-pyrimidine, in accordance with one embodiment of the present invention.

FIGS. 13a, 13b, 13c, 13d, 13e, 13f are clot formation and lysis profiles of various embodiments of the invention, hirudin and D-Phe-Pro-Arg chloromethylketone.

FIG. 14 is a plot of relative thrombin clot time of various concentrations of embodiments of the invention.

FIG. 15 is a plot of relative thrombin clot time of Hirudin and D-Phe-Pro-Arg chloromethylketone.

### DETAILED DESCRIPTION OF THE INVENTION

In one aspect, the invention provides novel disulphides of the general Formula I:

RSSR¹ (I)

wherein R is pyridine, phenylamine or pyrimidine, and R¹ is CH₃.

In another aspect, the present invention provides a process from preparing a disulphide of the general Formula I as described above, which comprises reacting a corresponding thiol with S-methyl methanethiolsulfonate in MeOH to obtain the title compound.

In yet another aspect, the present invention provides an anti-proliferative agent active against mammalian cells comprising as active ingredient at least one compound selected from the group of compounds consisting of:
4-methyldisulfanyl-pyridine (Formula II),
2-methyldisulfanyl-pyridine (Formula III),
4-methyldisulfanyl-phenylamine (Formula IV), and
2-methyldisulfanyl-pyrimidine (Formula V), and optionally together with conventional pharmaceutically acceptable ingredients.

### DEFINITIONS

“Alkyl” means linear and branched structures, and combinations thereof, and extends to cover carbon fragments having up to 20 carbon atoms. Examples of alkyl groups include octyl, nonyl, undecyl, dodecyl, tridecyl, tetradecyl, pentadecyl, eicosyl, 3,7-diethyl-2,2-dimethyl-4-propylnonyl, and the like.

“Lower alkyl” means alkyl groups of from 1 to 7 carbon atoms. Examples of lower alkyl groups include methyl, ethyl, propyl, isopropyl, butyl, sec- and tert-butyl, pentyl, hexyl, heptyl, and the like.

“Cycloalkyl” means a hydrocarbon containing one or more rings having from 3 to 12 carbon atoms, with the hydrocarbon having up to a total of 20 carbon atoms. Examples of cycloalkyl groups are cyclopropyl, cyclopentyl, cycloheptyl, adamantyl, cyclododecylmethyl, 2-ethyl-1-bicyclo[4.4.0]decyl and the like.

“Lower alkenyl” means alkenyl groups of 2 to 7 carbon atoms. Examples of lower alkenyl groups include vinyl, allyl, isopropenyl, pentenyl, hexenyl, heptenyl, 1-propenyl, 2-butenyl, 2-methyl-2-butenyl and the like.

“Lower alkoxy” means alkoxy groups of from 1 to 7 carbon atoms of a straight, branched, or cyclic configuration. Examples of lower alkoxy groups include methoxy, ethoxy, propoxy, isopropoxy, cyclopropyloxy, cyclohexyloxy, and the like.

“Alkylcarbonyl” means alkylcarbonyl groups of 1 to 20 carbon atoms of a straight, branched or cyclic configuration. Examples of alkylcarbonyl groups are 2-methylbutanoyl, octadecanoyl, 11-cyclohexylundecanoyl and the like. Thus, the 11-cyclohexylundecanoyl group is c-Hex-(CH₂)₁₀—C(O)—.

“Lower alkylcarbonyl” means alkylcarbonyl groups of from 1 to 8 carbon atoms of a straight, branched, or cyclic configuration. Examples of lower alkylcarbonyl group are formyl, 2-methylbutanoyl, cyclohexylacetyl, etc. By way of illustration, the 2-methylbutanoyl groups signifies —COCH(CH₃)CH₂CH₃.

“Lower alkylsulfonyl” means alkylsulfonyl groups of from 1 to 7 carbon atoms of a straight, branched, or cyclic configuration. Examples of lower alkylsulfonyl groups are methylsulfonyl, 2-butylsulfonyl, cyclohexylmethylsulfonyl, etc. By way of illustration, the 2-butylsulfonyl group signifies —S(O)₂CH(CH₃)CH₂CH₃.

Halogen means F, Cl, Br, and I.

It is intended that the definitions of any substituent (e.g., R, R¹, R², R⁶, etc.) in a particular molecule be independent of its definitions elsewhere in the molecule. Thus, NR⁶₂ represents —NHH, —NHMe, —N(Me)(Et), etc.

The heterocycles formed when two R⁶ (or R²⁰) groups join through N include pyrrolidine, piperidine, morpholine, thiamorpholine, piperazine, and N-methylpiperazine.

Optical Isomers—Diastereomers—Geometric Isomers

Some of the compounds described herein may contain one or more asymmetric centers and may thus give rise to diastereomers and optical isomers. The present invention is meant to comprehend such possible diastereomers as well as their racemic and resolved, enantiomerically pure forms and pharmaceutically acceptable salts thereof.

Salts

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary, and tertiary amines, substituted amines including naturally occurring substituted amines, cyclic amines and basic ion exchange resins, such as arginine, betaine, caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Mixed salts may at times be advantageous.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

It will be understood that in the discussion of methods of treatment which follows, references to the compounds of Formula I are meant to also include the pharmaceutically acceptable salts.

Compounds of Formula I of the present invention may be prepared according to the synthetic routes outlined in the Schemes 1 to 4 and by following the methods described herein. Following the schemata, Table 1 illustrates compounds representative of the present invention.

General Experimental Methods

Reagents were provided by Sigma-Aldrich Chemical Company and Oakwood Chemicals Company. Methanethiolsulfonates were provided by Toronto Research Chemicals. Reagents were used without further purification. Solvents were obtained from Aldrich and Caledon. Prior to the preparation of the disulphides, methanol was purged of oxygen by bubbling nitrogen an inert gas through it for several minutes. Thin-layer chromatography (TLC) was carried out using EM Reagent plates with fluorescence indicator (SiO2-60, F-254). Products were purified by flash chromatography, which were performed on silica gel H (200-300 mesh), and the solvent proportions were expressed on a volume/volume basis. 1H-NMR spectra were recorded using a Brucker 300 MHz in CDCl₃ unless otherwise noted.

### Synthesis of 4-methyldisulfanyl-pyridine is shown in Scheme 1

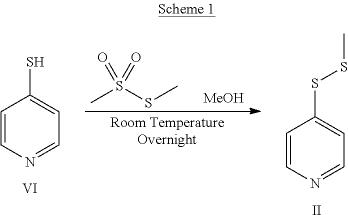

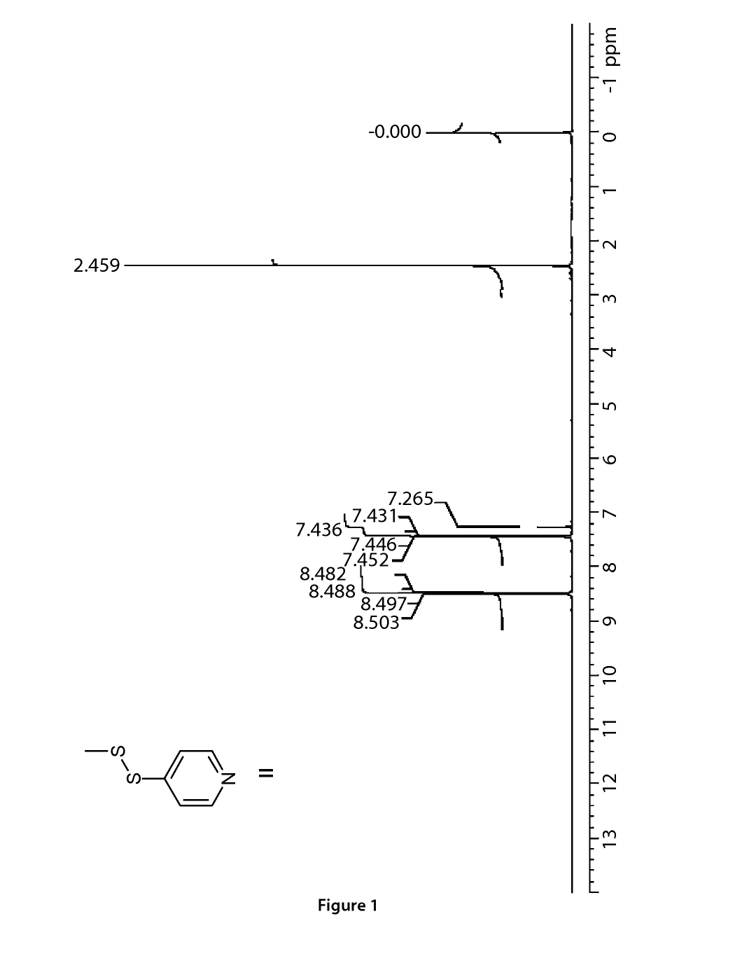

With reference to Scheme 1, to a solution of pyridine-4-thiol (VI) (700 mg, 6.296 mmol) in MeOH (4 ml) was added a solution of 1 equivalent of S-methyl methanethiolsulfonate (0.594 ml) in MeOH (1 ml). The yellow reaction mixture was stirred at room temperature under an argon atmosphere for 12 hrs. The mixture was then concentrated under reduced pressure to provide the crude product as yellow oil. The crude was taken up in ethyl acetate (2 ml), and passed through a column silica-gel chromatography using 100% ethyl acetate as eluant to give the desired product, 4-methyldisulfanyl-pyridine (II), 484 mg, 49% as light yellow oil. The 1H-NMR spectrum of the resulting compound, 4-methyldisulfanyl-pyridine (II), is shown in FIG. 1.

### Synthesis of 2-methyldisulfanyl-pyridine is shown in Scheme 2

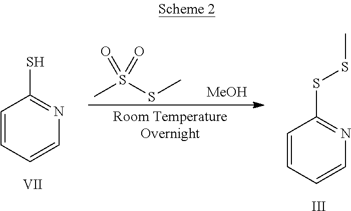

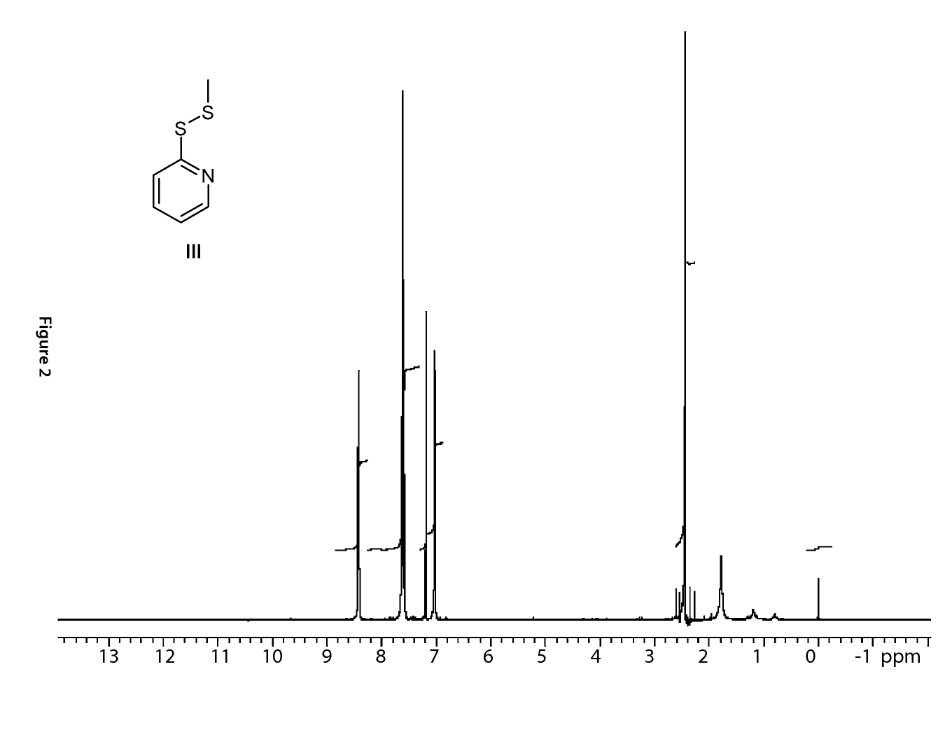

In a similar manner, with reference to Scheme 2, compound 2-methyldisulfanyl-pyridine (III) was obtained from pyridine-2-thiol (VII) as yellow oil. The 1H-NMR spectrum of the resulting product, 2-methyldisulfanyl-pyridine (III), is shown in FIG. 2.

### Synthesis of 4-methyldisulfanyl-phenylamine is shown in Scheme 3

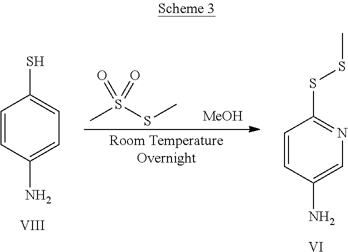

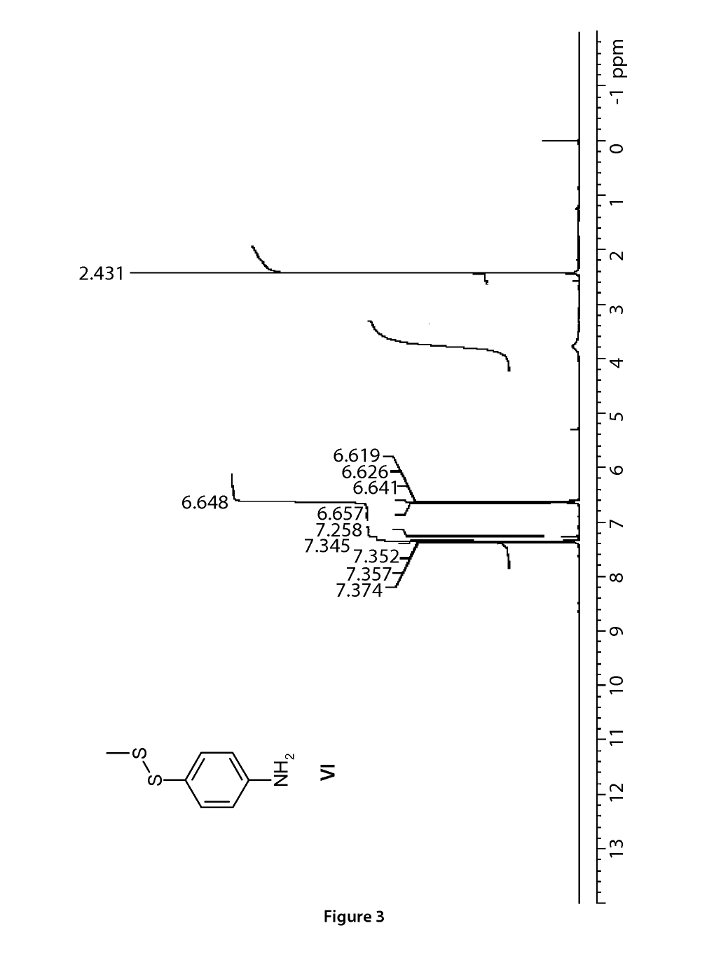

In a similar manner, with reference to Scheme 3, compound 4-methyldisulfanyl-phenylamine (VI) was obtained from 4-amino-benzenethiol (VIII) as yellow oil. The 1H-NMR spectrum of the resulting product, 4-methyldisulfanyl-phenylamine (VI), is shown in FIG. 3.

### Synthesis of 2-methyldisulfanyl-pyrimidine is shown in Scheme 4

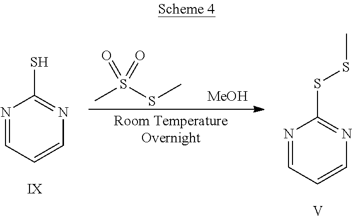

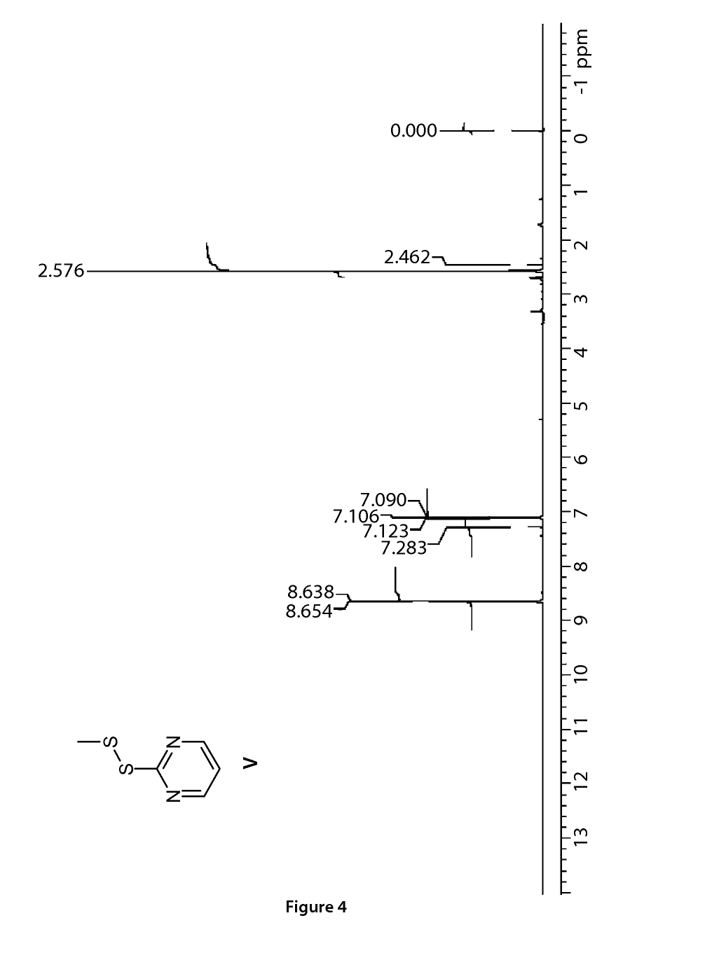

In a similar manner, with reference to Scheme 4, compound 2-methyldisulfanyl-pyrimidine (V) was obtained from pyrimidine-2-thiol (IX) as yellow oil. The 1H-NMR spectrum of the resulting product, 2-methyldisulfanyl-pyrimidine (V), is shown in FIG. 4.

TABLE 1
Compounds of the present invention

| | Molecular | Molecular weight |
|---|---|---|
| Compound | formula | (mol/g) |
| 4-methyldisulfanyl-pyridine (II) | C₆H₇NS₂ | 157.26 |
| 2-methyldisulfanyl-pyridine (III) | C₆H₇NS₂ | 157.26 |
| 4-methyldisulfanyl-phenylamine (IV) | C₇H₉NS₂ | 171.28 |
| 2-methyldisulfanyl-pyrimidine (V) | C₅H₆N₂S₂ | 158.24 |

The potential platelet inhibitory activity of the compounds listed in Table 1 was assessed using in vitro tests of platelet function. Further, it was determined whether these compounds effect thrombin-mediated conversion of fibrinogen to fibrin and the subsequent degradation of a fibrin by tissue plasminogen activator (t-PA). Stock solutions of each compound were prepared in DMSO. Subsequent dilutions of drug were made in non-buffered saline containing 10% (v/v) DMSO.

Platelet Inhibition Assays

Platelet function studies were performed simultaneously in both whole blood (using whole blood impedance platelet aggregometry with a Multiplate System) and platelet rich plasma (light transmission aggregometry with a Chrono-Log System). Blood was collected from healthy individuals into vacutainers containing 3.2% (w/v) citrate and was used within 3 hours of collection. Platelet rich plasma (“PRP”) was prepared by centrifugation at 170 g for 15 minutes, and the platelet count measured and adjusted to 250×109/L for use in the light transmission aggregometry studies.

Each compound was tested at final concentrations of 10, 25, 50 and 100 μM. For each test, the compound was diluted 1/100 into the PRP or whole blood to obtain the final concentrations listed. The PRP or whole blood sample was allowed to stir in the aggregometer for 3 minutes prior to agonist addition. One compound was tested each day.

As a positive control to verify the sensitivity to inhibition of thrombin receptor agonist peptide (TRAP)-mediated aggregation, TRAP-induced aggregations were also done in the presence of eptifibatide, a glycoprotein IIb/IIIa inhibitor (final concentration of 77 μg/mL in PRP and 65 μg/mL in whole blood).

Materials used included:
The four channel light transmission aggregometer instrument used for the platelet rich plasma studies was from Chrono-Log Corporation.
The ADP and collagen agonists used for platelet rich plasma studies were from Chrono-Log Corporation.
The four channel Multiplate whole blood impedance platelet aggregometer (IPA) was from DiaPharma Group.
The ADP and collagen agonists used for the whole blood studies were provided by DiaPharma Group. The TRAP agonist used in both the whole blood and PRP studies was also obtained from DiaPharma Group.

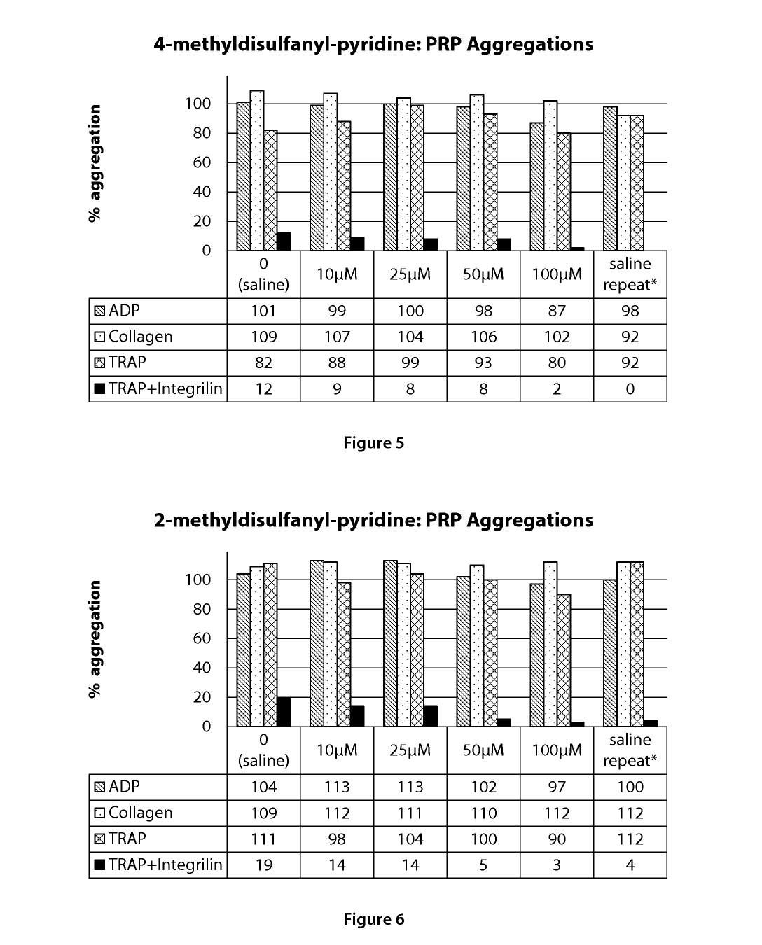

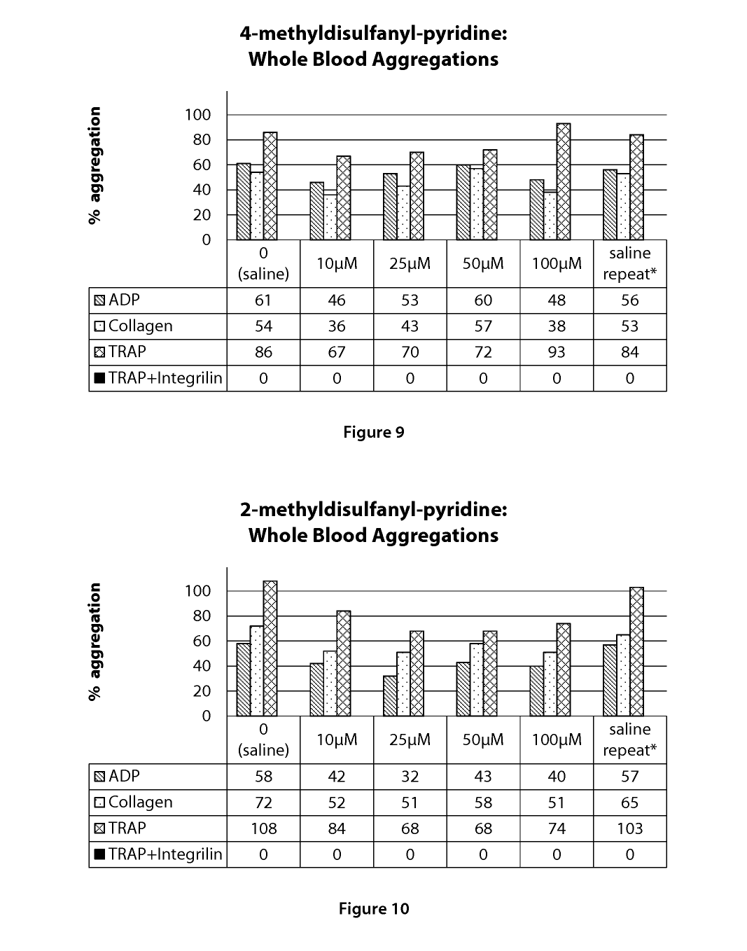

Results for PRP aggregations are shown in FIGS. 5, 6, 7 and 8 for compounds 4-methyldisulfanyl-pyridine (II), 2-methyldisulfanyl-pyridine (III), 4-methyldisulfanyl-phenylamine (VI), and 2-methyldisulfanyl-pyrimidine (V), respectively. Results for whole blood aggregations are shown in FIGS. 9, 10, 11 and 12 for compounds 4-methyldisulfanyl-pyridine (II), 2-methyldisulfanyl-pyridine (III), 4-methyldisulfanyl-phenylamine (VI), and 2-methyldisulfanyl-pyrimidine (V), respectively.

Referring to FIGS. 9, 10, 11 and 12, little anti-platelet effect was observed under the conditions used, with the surprising exception of 2-methyldisulfanyl-pyrimidine (V) in FIG. 12. Also, when comparing the results shown in FIGS. 5, 6, 7 and 8, the whole blood aggregation method appears to be more sensitive to the inhibitory effects where observed.

Referring to FIGS. 5 and 9, in one embodiment, 4-methyldisulfanyl-pyridine (II) shows no inhibitory activity observed in PRP with any agonist tested. However, evident is a reduction in response to all agonists in whole blood, but this response does not appear to be dose dependent.

Referring to FIGS. 6 and 10, in one embodiment, 2-methyldisulfanyl-pyridine (III) shows no inhibitory activity observed in PRP with any agonist tested. However, evident is approximately a 25% reduction in response to all agonists in whole blood, but again, this response does not appear to be dose dependent.

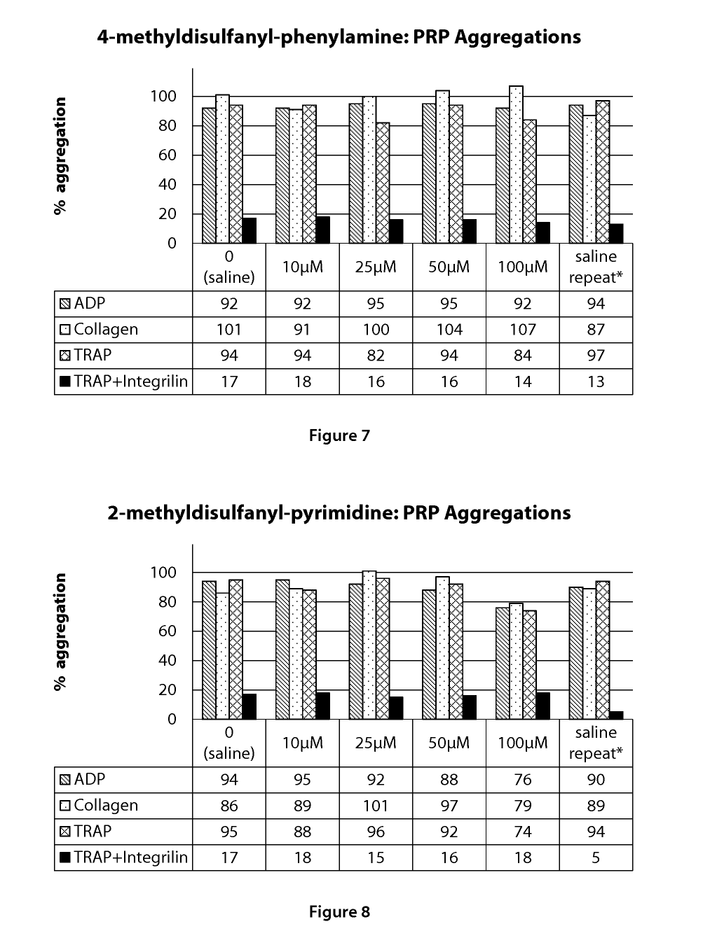

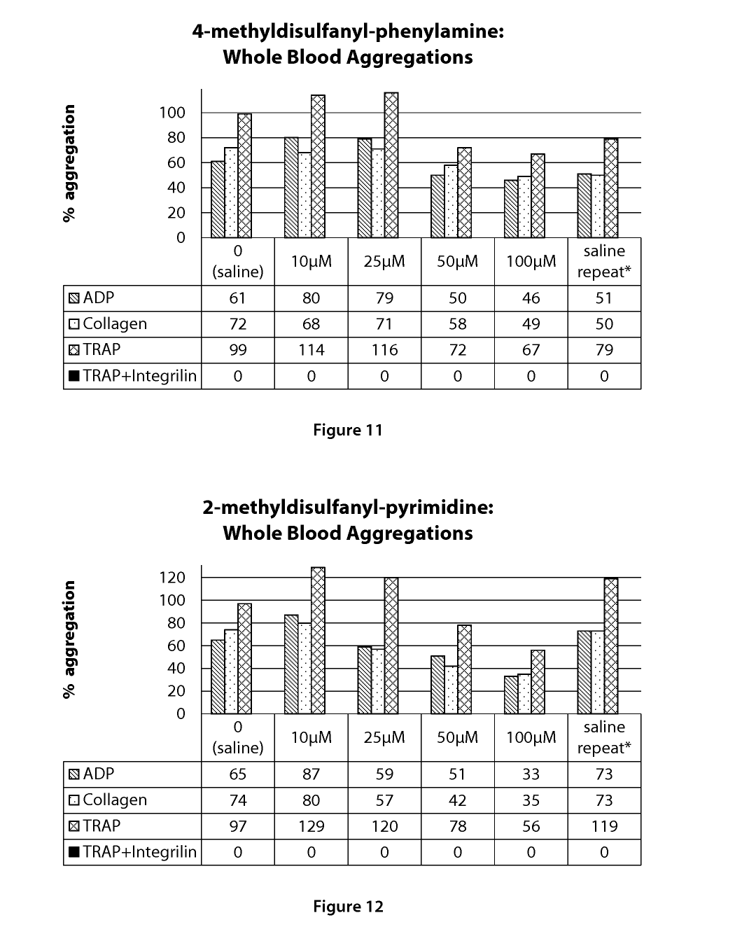

Referring to FIGS. 7 and 11, in one embodiment, 4-methyldisulfanyl-phenylamine (IV) shows no inhibitory activity observed in PRP with any agonist tested. Some reduction in response to all agonists is shown in whole blood, but this response does not appear to be dose dependent.

Referring to FIGS. 8 and 12, in one embodiment, 2-methyldisulfanyl-pyrimidine (V) shows surprising inhibitory activity observed in PRP with all agonists at the highest concentration tested (100 μM). Moreover, a positive dose dependent inhibition of platelet response to all agonists was observed in whole blood. At 100 μM, 2-methyldisulfanyl-pyrimidine (V) appeared to reduce platelet response to all agonists by approximately 50%, as shown in FIG. 12.

Coagulation/Fibrinolysis Assays

Coagulation and fibrinolysis were studied simultaneously in a 96-well microtiter plate-based assay system. Varying concentrations of drug in 50 μl buffer or an equivalent volume of saline were added to the wells. To this was added 100 μl of 6 μM fibrinogen, 1 μM Glu-plasminogen and 4 mM CaCl₂. Reactions were initiated by addition of 50 μl of buffer containing 2 nM t-PA and 20 nM thrombin. Final concentrations of reagents in the wells were 3 μM fibrinogen, 500 nM Glu-plasminogen, 2 mM CaCl₂, 0.05 nM t-PA, 5 nM thrombin and compounds of Formulas (II), (Ill), (IV) or (V) (0-400 μM). As positive controls, hirudin or D-Phe-Pro-Arg chloromethylketone (PPACK) was used in place of the subject compounds. Plates were incubated at 23° C. for 1 h with constant shaking and turbidity was monitored continuously at 405 nm. Clot times and lysis times were determined using instrument soft wave as the times to half maximal increase and decrease in turbidity, respectively. Data are expressed as the mean±SEM of three experiments, each done in triplicate.

Materials used:
Fibrinogen and thrombin were from Enzyme Research Laboratories and t-PA was from Genentech.
Glu-plasminogen was isolated from human plasma as we have described.
Hirudin and PPACK were from Behring AG and Calbiochem, respectively.
The plate reader was from Molecular Devices.

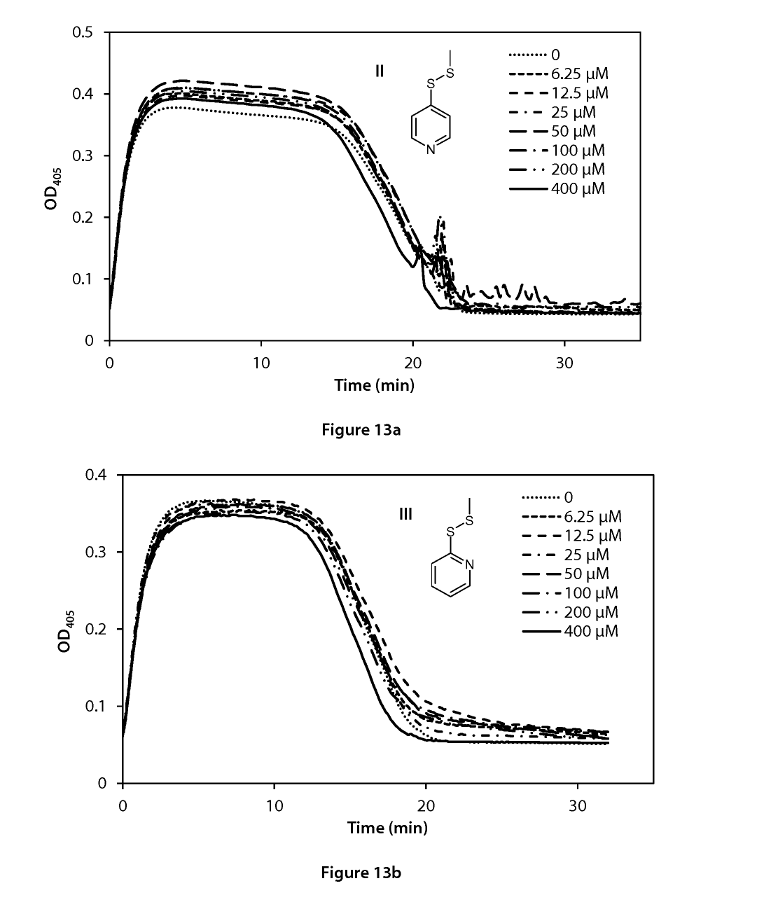

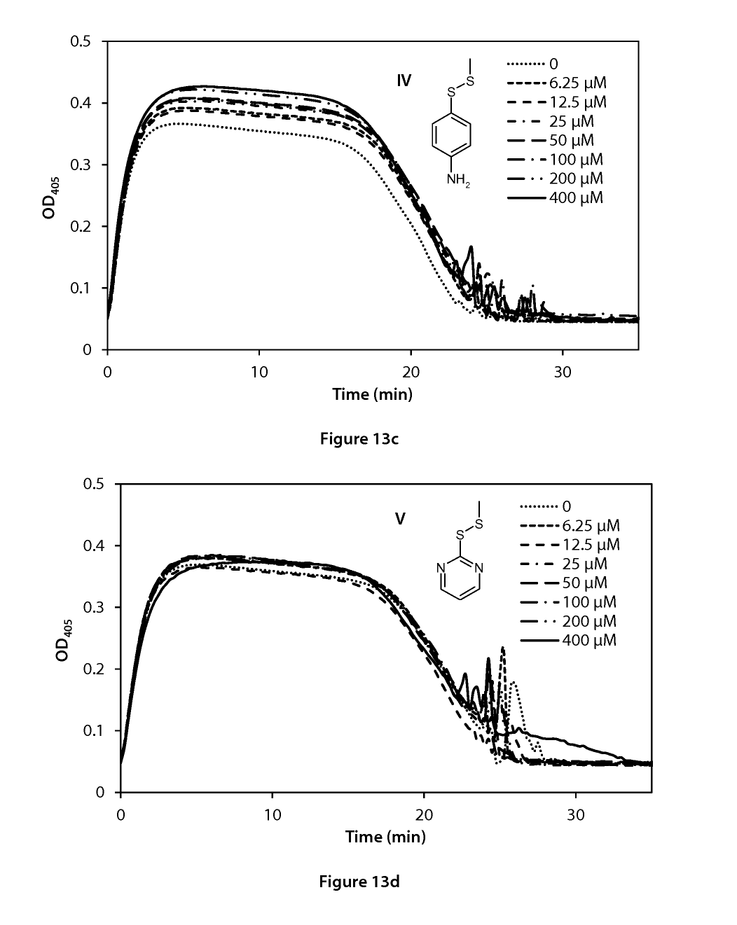

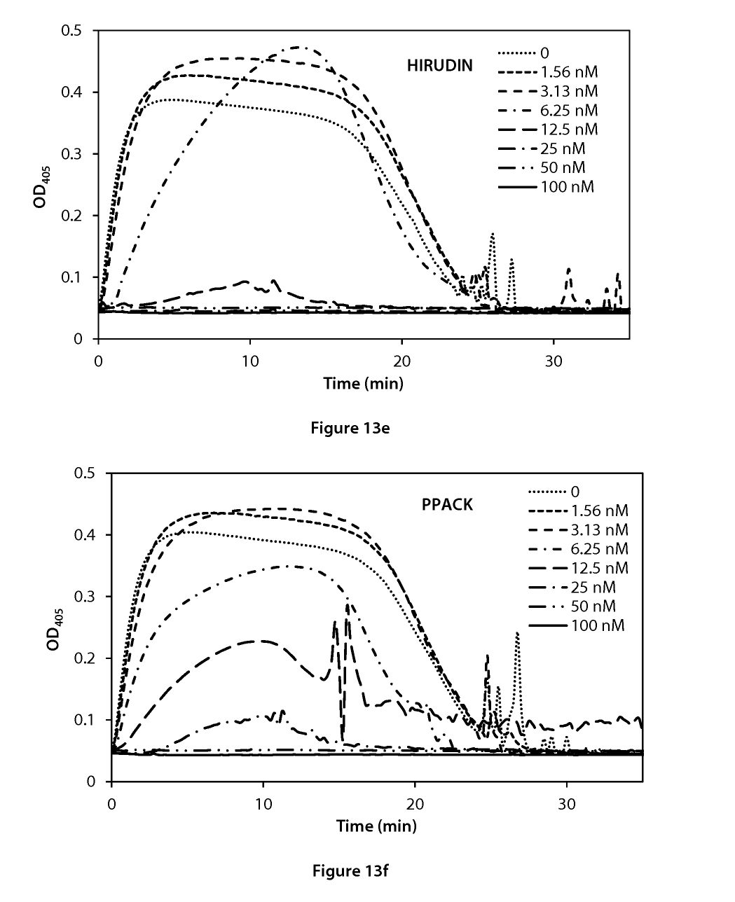

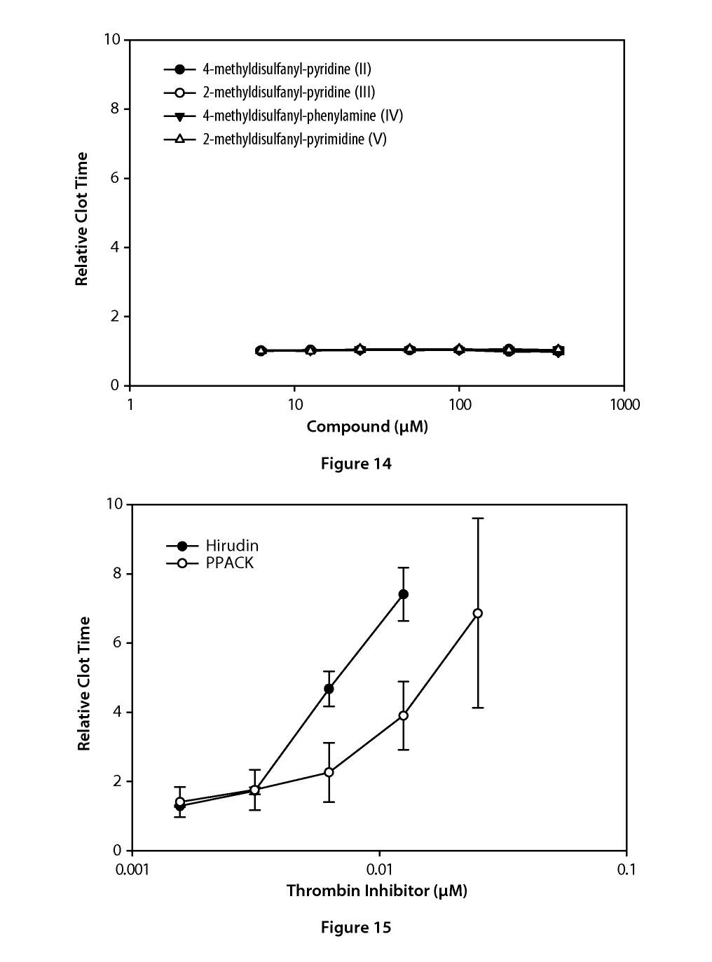

Results for clot formation and clot lysis are shown in FIGS. 13a, 13b, 13c, 13d, 13e, 13f for compounds 4-methyldisulfanyl-pyridine (II), 2-methyldisulfanyl-pyridine (III), 4-methyldisulfanyl-phenylamine (IV), and 2-methyldisulfanyl-pyrimidine (V), hirudin and PPACK, respectively. Relative clot times for compounds 4-methyldisulfanyl-pyridine (II), 2-methyldisulfanyl-pyridine (III), 4-methyldisulfanyl-phenylamine (IV), and 2-methyldisulfanyl-pyrimidine (V) are shown in FIG. 14. Relative clot times for hirudin and PPACK are shown in FIG. 15.

Referring to FIGS. 13a, 13b, 13c, 13d, the plots of the clot formation and lysis profiles, increasing concentration of compounds of the present invention had no effect on the profiles when used at concentration up to 400 μM. In contrast, both hirudin and PPACK inhibited clot formation and lysis, as shown in FIGS. 13e, 13f.

Each compound of the present invention was tested at concentrations of 0-400 μM and was found to have no effect on the thrombin clotting time. In contrast, when hirudin or PPACK was tested at concentrations of 0-100 nM, it caused complete inhibition of clotting at 25 nM and 50 nM, respectively, with a measurable effect seen as low as 6.25 nM.

Referring to FIG. 14, none of the compounds of the present invention had an effect on the clot lysis times. Referring to FIG. 15, with concentrations of hirudin or PPACK, lysis times were shortened. Accordingly, the compounds of the present invention have no effect on thrombin-mediated conversion of fibrinogen to fibrin or on the subsequent degradation of fibrin by t-PA.

1. A compound of Formula (I)
RSSR¹ (I)
or a pharmaceutically acceptable salt form thereof, wherein R is selected from pyridine, phenylamine and pyrimidine, and R¹ is CH₃.

2. The compound of claim 1, selected from the group: 4-methyldisulfanyl-pyridine, 2-methyldisulfanyl-pyridine, 4-methyldisulfanyl-phenylamine, and 2-methyldisulfanyl-pyrimidine.

3. A pharmaceutical composition comprising a pharmaceutically acceptable carrier and a therapeutically effective amount of a compound of Formula (I)
RSSR¹ (I)
or a pharmaceutically acceptable salt form thereof, wherein R is selected from pyridine, phenylamine and pyrimidine, and R¹ is CH₃.

4. The composition of claim 3, wherein the composition is provided as an anti-proliferative agent active against mammalian cells.

5. The composition of claim 3, wherein the composition is provided in an oral dosage form.

6. A method of treating a condition, wherein said method comprises administering to a host in need of such treatment a therapeutically effective amount of a compound of claim 1.

7. The method of claim 6, wherein the condition is selected from the group comprising: a blood platelet aggregation-related condition, thrombosis, inflammation, bone degradation, malignancy, a cell aggregation-related condition, thromboembolic disorders selected from thrombus or embolus formation, harmful platelet aggregation, re-occlusion following thrombolysis, reperfusion injury, restenosis, atherosclerosis, stroke, heart attack, peripheral arterial ischemia, myocardial infarction, and unstable angina.

8. A method for manufacturing a compound selected from the group comprising: 4-methyldisulfanyl-pyridine, 2-methyldisulfanyl-pyridine, 4-methyldisulfanyl-phenylamine, and 2-methyldisulfanyl-pyrimidine, wherein a precursor is reacted with S-methyl methanethiolsulfonate in solution with MeOH to form the desired compound.

9. The method for manufacturing of claim 8, wherein pyrimidine-2-thiol is reacted with S-methyl methanethiolsulfonate in solution with MeOH to form 2-methyldisulfanyl-pyrimidine.
